(19) United States
(12) Patent Application Publication
(10) Pub. No.: US 2008/0182933 A1
(43) Pub. Date: Jul. 31, 2008

(54) Filler for Porous Film and Porous Film Containing the Same

(76) Inventors: Seiya Shimizu, Hyogo (JP); Makoto Nagamatsu, Hyogo (JP); Hiroshi Shibata, Hyogo (JP); Shoji Ehara, Hyogo (JP)

Correspondence Address: KRATZ, QUINTOS & HANSON, LLP, 1420 K Street, N.W., Suite 400, WASHINGTON, DC, 20005, US

(21) Appl. No.: 11/792,523

(22) PCT Filed: Dec. 9, 2005
(86) PCT No.: PCT/JP2005/022649

§ 371 Date: Jun. 7, 2007

(30) Foreign Application Priority Data
Dec. 17, 2004 (JP) 2004-365544
Mar. 25, 2005 (JP) 2005-089057

(51) Int. Cl. A61K 9/16

(52) U.S. Cl. 524/543

(57) ABSTRACT

A filler for porous films is provided which is easy to be mixed with a resin, has good dispersibility in a resin, and provides a porous film useful for a light reflector of, for example, a liquid crystal display and a lighting apparatus and a porous film useful for a diaphragm (separator) between electrodes of a battery.

A filler comprises inorganic particles surface treated with a surfactant (A) and a compound (B) having a chelating function to an alkaline earth metal.

### TECHNICAL FIELD

The present invention relates to a filler for porous films which is easy to be mixed with a resin, excellent in dispersibility in a resin and comprises surface-treated inorganic particles scarcely containing impurities or coarse particles and a porous film containing the filler.

More particularly, the present invention relates to a filler useful for giving excellent properties to a porous film; that is, the filler is useful for giving a porous film with high resistance to strength deterioration since the filler has good workability at the time of preliminary mixing with a resin or other additives, has good dischargeability and scarcely cuts molecular chains (deteriorates molecules) of a resin at the time of melting and blending, hardly causes re-agglomeration of its particles among themselves or with other additives and resins, and scarcely contains impurities and coarse particles. Further, since it is possible to adjust the particle diameter of the filler and to disperse the filler extremely evenly in a film, the filler is useful for giving a porous film having a void diameter within a controlled and constant distribution width. The present invention also relates to a porous film containing the filler.

### BACKGROUND ART

Porous films made of synthetic resins have been used for various purposes and applications such as synthetic paper, sanitary materials, medical materials, construction materials, air-permeable sheets for agriculture, light reflectors for liquid crystal displays, separators for various kinds of batteries, and further improvement and development of the porous films have been desired in all of the application fields.

For example, a transmissive type liquid crystal display has been used as a monitor for a personal computer and a display apparatus of a flat panel TV and in such a liquid crystal display, a panel type illumination apparatus, so-called backlight, is generally installed in the rear side of a liquid crystal device.

Further, since a secondary lithium battery used for mobile appliances such as cellular phones and notebook personal computers has a high energy density for the volume and the weight as compared with other batteries, its production and consumption have been increasing at a high rate since the secondary lithium battery was put to practical use in the beginning of 1990s.

Along with further advancement of the functions of various kinds of mobile appliances, it has been required for a secondary lithium battery as a main power source thereof to have further improved properties, and similarly to both positive and negative electrodes, the separator is also required to have improved properties.

A backlight has a function of converting a linear light source such as a cold cathode discharge tube into a panel-like light source and as a typical structure, there are a type of a light source to be installed immediately under rear side of a liquid crystal device and a type (side light type) which gives a panel-like light source by scattering light of a linear light source from a side face to a plane-like form through a transmissive light guide body made of an acrylic plate or the like.

To satisfy recent requests from consumers for lightweight and thinness of displays, a side light type display apparatus in which the backlight unit can be thin in terms of the structure is preferred and has been employed more in liquid crystal display apparatuses such as a portable personal computer.

The side light type backlight unit typically comprises a light guide made of an acrylic plate or the like, a light reflector made of a foamed polyester or polyolefin film, a metal-evaporated film or the like, a light diffuser to be installed on the opposite face of the light reflector, and a cold cathode discharge tube installed in the side face of the light guide.

Dot printing with a reflective coating material is carried out on the surface of the light guide facing the light reflector side and linear light led from the side face of the light guide emits luminescence at the dot printing parts and becomes evenly planer light in the diffuser in combination with the light reflected by the light reflector.

Functions required to the light reflector in the backlight unit are efficient utilization of the light from a built-in light source, long life with little change in light reflectivity and color tone, and display satisfying the needs of consumers.

That is, it is required to evenly reflect light which is transmitted to the light reflector side from the light guide to the plane direction without vain and unevenness of the brightness, and in these days when the liquid crystal color display becomes common, it is required for a light source of a color liquid crystal cell, which is a main device of various kinds of liquid crystal displays, to have sufficient brightness since the light transmittance of the color liquid crystal cell is low. In addition, naturally, it is required to have sufficient brightness with little color tone alteration.

Further, since consumers generally dislike dazzling specular reflection, it is needed to achieve light emission with relatively uniform brightness in the emitting direction by diffused reflection and make consumers feel natural the light from a display.

To deal with the physical properties required to the light reflector described above, white polyester films (e.g., refer to Patent Document 1) have been used and porous polyolefin films for improving the color tone alteration of the white polyester films have been proposed (e.g., refer to Patent Documents 2 and 3).

On the other hand, a secondary lithium battery comprises both positive and negative electrodes and their lead wires, a porous film separator having through holes for allowing a lithium ion to move in and out while preventing short circuit between both electrodes at the time of charging and discharging, an organic solvent (an electrolytic solution) as a lithium ion transportation medium with which the separator is impregnated, and a metal container for packaging to prevent leakage of the electrolytic solution.

To obtain a battery with a high capacity, it is more desirable for the battery that the surface areas of both electrodes are wider and an ion moves easier between both electrodes. Generally, with respect to a lithium battery, a wider efficient electrode surface area is obtained by laminating a thin film-like positive electrode, a separator, and a negative electrode and rolling the laminated unit.

In addition to insulating property between both electrodes, which is an intrinsic purpose of the separator, the separator is desired to be thin and have high porosity and high ventilation property since the inner resistance is lowered more and the capacity of the battery is improved more as ion permeability of the separator is higher.

However, the insulating property and the decrease of the inner resistance which are functions of the separator are mutually contradictory and it is not sufficient to make the separator simply thin, and the size stability, corrosion resistance to an electrolytic solution, workability at the time of rolling, and cost should be considered for the separator. Additionally, today the separator is required to have a shut down function for stopping a battery reaction by melting the resin and clogging holes in order to assure safety in the case an abnormal current is generated because of erroneous connection and temperature in the inside of the battery is increased.

In relation to the above-mentioned requests, however, in the practical situation, types of usable resins and thickness of films are limited for each purpose.

So far, porous films employed as materials for sanitary goods such as diapers and bed covers and clothing such as gloves have been used for separators for secondary lithium batteries.

However, porous films more suitable for the requests have been investigated and developed and for example, a method for obtaining a porous film by blending resin particles with an average particle diameter of 0.01 to 10 μm and a β-nucleating agent with polypropylene, forming a film using the obtained polypropylene composition, and rolling and stretching the film was proposed (e.g., refer to Patent Document 4).

Further, taking the size of pores of a porous film into consideration in terms of the inner resistance of a battery, also proposed is a film for separators having a low inner resistance by adding a prescribed amount of inorganic particles with an average particle diameter of 1 μm or smaller to a thermoplastic resin, producing a primary film using the mixture, and controlling the size of the pores of the porous film by stretching the primary film in specified conditions (e.g., refer to Patent Document 5).

Patent Document 1: Japanese Patent Application Laid-Open (JP-A) No. 04-239540

Patent Document 2: JP-A No. 2002-31704

Patent Document 3: JP-A No. 2004-157409

Patent Document 4: JP-A No. 9-176352

Patent Document 5: JP-A No. 2002-201298

### DISCLOSURE OF THE INVENTION

### Problems to be Solved by the Invention

However, because of remarkable development of recent IT technologies, it is required not only to make a display apparatus large in the plane, lightweight and thin in the thickness direction but also to improve the preciseness of the pixels of the display, and also the brightness of the light emitted from a light source of a backlight is required to be higher and more stable with the lapse of time.

With respect to the white polyester film of Patent Document 1, since the resin near a light source was sometimes deteriorated and discolored because of heat emitted from the light source and light rays with wavelengths close to the wavelength of ultraviolet rays, the color tone of a liquid crystal display was sometime changed or deteriorated with the lapse of time.

Particularly, since the light source itself was made powerful and the distance to the light source was shortened because of the request for higher brightness, the resin was deteriorated more remarkably and accordingly higher stability with the lapse of time was desired.

Patent Documents 2 and 3 disclose that polyolefin type resins, which are said to be more resistant to deterioration with the lapse of time than polyester resins, and inorganic particles of heavy calcium carbonate and barium sulfate as particles for forming fine pores in the resins are used to obtain films which are more stable with the lapse of time, scarcely decrease in brightness, contain flexible resins, and hardly form scratches on a light guide plate.

However, the above-mentioned methods were insufficient to satisfy the request for higher brightness to the liquid crystal display apparatus in these days and therefore further improvements were required.

On the other hand, with respect to a separator film, conventional porous films are not only incapable of and unsuitable for satisfying high capacity and high output power but also insufficient for large scale batteries and automotive batteries whose development is highly expected in the future, and further improvements are required.

For example, in the case of a lithium battery using the porous film obtained by the above-mentioned method of Patent Document 4 as a separator, although the reasons are not clear, the inner resistance of the battery was increased and output obtained by improvement of both positive and negative electrodes was consumed in vain and thus the porous film was not satisfactory as a separator film.

Further, if a porous film was produced by the method described in Patent Document 5, the film had many portions where the insulation was failed supposedly because of uneven particle size of the particles used for the film, and in terms of the inspection and removal of the defection portions and the yield, the cost of the film formation was increased and also the inner resistance of the obtained battery was undesirably increased.

As a present method for producing a film having pores, there are a method for forming pores, so-called voids, between inorganic particles and a resin by mixing the particles and the resin and carrying out a uniaxial or biaxial stretching process, and a method for dissolving the particles with an acid or an alkali. In both methods, it is required to obtain a porous film with little dispersion of the size of the voids or pores formed therein and even distribution of the voids in the film plane. Therefore, it is required that the inorganic particles are evenly dispersed in a resin composition for films, scarcely contain impurities or coarse particles, which sometimes induce short circuit between both electrodes, and thus have sharp particle size distribution.

Further, as described above, since present lithium ion batteries are required to have a shut down function, polyolefin type resins with a low melting point are used, and accordingly it is required that inorganic particles are blended economically and easily with the resins.

In view of the above state of the art, it is an object of the present invention to provide a filler for porous films which is easy to be mixed with a resin to be a porous film substrate, excellent in dispersibility in the resin and comprises surface-treated inorganic particles scarcely containing impurities or coarse particles, and a porous film containing the filler. It is an object of the present invention to provide a filler useful for giving excellent properties to a porous film and a porous film containing the filler; that is, the filler is useful for giving a porous film with high resistance to strength deterioration since the filler has good workability at the time of preliminary mixing with a resin or other additives, has good dischargeability and scarcely cuts molecular chains (deteriorates molecules) of a resin at the time of melting and blending, hardly causes re-agglomeration of its particles among themselves or with other additives, and scarcely contains impurities and coarse particles, and further, since it is possible to adjust the particle diameter of the filler and to disperse the filler extremely evenly in a film, the filler is useful for giving a porous film having a void diameter within a controlled and constant distribution width.

### Means for Solving the Problems

The present inventors have made various investigations to solve the above-mentioned problems and have found that it is made possible to obtain surface-treated inorganic particles remarkably excellent in dispersibility in a resin, to make it easy to blend the obtained surface-treated inorganic particles with a resin, and to carry out dispersion of the surface-treated inorganic particles without causing re-agglomeration by using a surfactant in combination with a compound having a chelating function to alkaline earth metals as a surface treatment agent. Further, the present inventors have found that in the case a resin composition for porous films which contains the surface-treated inorganic particles is used for forming a film stretched uniaxially or biaxially, good voids are formed and the obtained film is useful as a film for a light reflector of a backlight apparatus such as a liquid crystal display and also useful as a separator for a secondary lithium battery. Accordingly, these findings now led to completion of the present invention.

That is, claim 1 of the present invention is a filler for porous films comprising inorganic particles surface treated with a surfactant (A) and a compound (B) having a chelating function to an alkaline earth metal.

Claim 2 of the present invention is the filler for porous films according to claim 1, wherein the inorganic particles are calcium carbonate or barium sulfate.

Claim 3 of the present invention is the filler for porous films according to claim 1, wherein the surfactant (A) is a fatty acid salt.

Claim 4 of the present invention is the filler for porous films according to any one of claims 1 to 3, wherein the surfactant (A) has a composition containing 50 to 98% by weight of a linear fatty acid salt having 16 or higher carbon atoms and 1.5 to 50% by weight of a linear fatty acid salt having 10 to 14 carbon atoms.

Claim 5 of the present invention is the filler for porous films according to any one of claims 1 to 4, wherein condensed phosphoric acid of the compound (B) having the chelating function to an alkaline earth metal is a cyclic condensed phosphoric acid or metaphosphoric acid.

Claim 6 of the present invention is the filler for porous films according to any one of claims 1 to 5, wherein the ratios of the surfactant (A) and the compound (B) having a chelating function to an alkaline earth metal to the inorganic particles are 0.1 to 20% by weight and 0.05 to 7% by weight, respectively.

Claim 7 of the present invention is the filler for porous films according to any one of claims 1 to 6 which satisfies the following particle size properties (1) to (4):

0.3≦D₅₀≦1.5 [μm]; (1)

0.02≦Dₓ≦0.6 [μm]; (2)

Dₐ≦20 [μm]; and (3)

3≦Sw≦60 [m²/g] (4)

wherein

D₅₀: average particle diameter [μm] of on-sieve particles in cumulative distribution measured by Microtrac FRA manufactured by Leeds & Northrup;

Dₓ: average particle diameter [μm] of particles left after observation of randomly selected 100 particles using a scanning electron microscope at 20,000 times magnification and elimination of the maximum and minimum 20 particles each among the particles;

Dₐ: maximum particle size [μm] observed in the case of measurement by Microtrac FRA manufactured by Leeds & Northrup; and

Sw: BET specific surface area [m²/g] measured by a nitrogen adsorption method.

Claim 8 of the present invention is a porous film containing the filler according to any one of claims 1 to 7.

Claim 9 of the present invention is the porous film according to claim 8, wherein the resin of the porous film is a polyolefin type resin.

Claim 10 of the present invention is the porous film according to claim 8 or 9 to be used for light reflection.

Claim 11 of the present invention is the porous film according to any one of claims 8 to 10 to be used for a light reflector for a liquid crystal display apparatus or a lighting apparatus.

Claim 12 of the present invention is the porous film according to claim 8 or 9 to be used for a separator between electrodes of a battery.

Claim 13 of the present invention is the porous film according to claim 12, wherein the battery is a secondary lithium battery.

### Effects of the Invention

A filler for porous films of the present invention is easy to be mixed with a resin with good dispersibility in the resin and suitable for providing a porous film useful as a light reflector of, for example, a backlight apparatus for a liquid crystal display and as a separator between electrodes for a battery. Further, the filler for porous films of the present invention can be mixed speedily with a resin and in addition has characteristics, that is, scarce adhesion to inner wall faces of a mixer and mixing and stirring blades, little deformation of resin induced by the adhesion in the inside of the mixer and generation of agglomerates, good workability of mixing, and slight occurrence of clogging of a strainer in post-treatment by a kneading extruder.

### BRIEF DESCRIPTION OF THE DRAWING

FIG. 1 is a schematic drawing of an immediately under type backlight unit employed for evaluation of brightness unevenness.

### EXPLANATION OF SYMBOLS

| | |
|---|---|
| 1 | housing |
| 2 | cold cathode lamp |
| 3 | LCD cell |
| 4 | light reflector |

### BEST MODE FOR CARRYING OUT THE INVENTION

Examples of a surfactant (A) to be used in the present invention are surfactants of saturated fatty acids, unsaturated fatty acids, alicyclic carboxylic acids, resin acids, their salts and their esters; alcohol type surfactants; sorbitan fatty acid esters; amide type surfactants; amine type surfactants; polyoxyalkylene alkyl ethers; polyoxyethylene nonyl phenyl ether; sodium α-olefinsulfonate; long chain alkylamino acids; amine oxides; alkylamines; and quaternary ammonium salts, and they may be used alone or if necessary, two or more of them may be used in combination.

Examples of the saturated fatty acids are capric acid, lauric acid, myristic acid, palmitic acid, and stearic acid: examples of the unsaturated fatty acids are oleic acid, linoleic acid, and linolenic acid: examples of alicyclic carboxylic acids are naphthenic acids containing carboxylic group at the terminal of cyclopentane ring or cyclohexane ring: and examples of resin acids are abietic acid, pimaric acid, and neoabietic acid.

Examples of the alcohol type surfactants are alkyl sulfuric acid ester sodium salt, alkyl ether sulfuric acid ester sodium salt: examples of the sorbitan fatty acid esters are sorbitan monolaurate and polyoxyethylene sorbitan monostearate: examples of the amide type or amine type surfactants are fatty acid alkanol amides and alkylamine oxides: examples of the polyoxyalkylene alkyl ethers are polyoxyethylene alkyl ethers and polyoxyethylene lauryl ethers: examples of the long chain alkylamino acids are lauryl betaine and stearyl betaine.

Examples of the amine oxides are polyoxyethylene fatty acid amides and alkylamine oxides: examples of the alkylamines include stearyl amine acetate. Examples of the quaternary ammonium salts are stearyltrimethylammonium chloride and quaternary ammonium sulfates.

Examples of the salts of the above-mentioned various kinds of acids are alkali metal salts such as potassium and sodium salts and practical examples are saturated fatty acid salts such as potassium laurate, potassium myristate, potassium palmitate, sodium palmitate, potassium stearate, and sodium stearate; unsaturated fatty acid salts such as potassium oleate and sodium oleate; alicyclic carboxylic acid salts such as lead naphthenate and lead cyclohexylbutylate; potassium or sodium abietate.

Further, examples of the above-mentioned esters of various kinds of acids are saturated fatty acid esters such as ethyl caproate, vinyl caproate, diisopropyl adipate, ethyl caprylate, allyl caprylate, ethyl caprylate, vinyl caprylate, diethyl sebacate, diisopropyl sebacate, cetyl isooctanoate, octyldodecyl dimethyloctanoate, methyl laurate, butyl laurate, lauryl laurate, methyl myristate, isopropyl myristate, cetyl myristate, myristyl myristate, isocetyl myristate, octyldodecyl myristate, isotridecyl myristate, methyl palmitate, isopropyl palmitate, octyl palmitate, cetyl palmitate, isostearyl palmitate, methyl stearate, butyl stearate, octyl stearate, stearyl stearate, cholesteryl stearate, isocetyl isostearate, methyl behenate, and behenyl behenate; and unsaturated fatty acid esters such as methyl oleate, ethyl linoleate, isopropyl linoleate, ethyl olive oleate, and methyl erucate, and also include heat resistant special fatty acid esters such as long chain fatty acid higher alcohol esters, neopentyl polyol (including long chain and middle chain) fatty acid type esters and partial ester compounds; dipentaerythritol long chain fatty acid esters, complex middle chain fatty acid esters, isocetyl 12-stearoylstearate, isostearyl 12-stearoylstearate, stearyl 12-stearoylstearate, octyl beef tallow fatty acid esters, and polyhydric alcohol fatty acid alkyl glyceryl ether fatty acid esters; and aromatic esters represented by benzoic acid esters.

The above-mentioned surfactants may be used alone or if necessary two or more of them may be used in combination.

The inorganic particles surface treated with the respective salts of saturated fatty acids, unsaturated fatty acids, alicyclic carboxylic acids, and resin acids among the above-mentioned surfactants are excellent in dispersibility without interfering insulation property or heat resistance of a resin when being mixed with the resin and particularly, a mixture of fatty acid alkali metal salts are preferable.

With respect to the saturated fatty acid alkali metal salts, it is preferable that the mixture has a composition containing 50 to 98% by weight of alkali metal salts of linear fatty acids having 16 or more carbon atoms such as palmitic acid, stearic acid, arachic acid, and behenic acid and 1.5 to 50% by weight of alkali metal salts of linear fatty acids having 10 to 14 carbon atoms such as capric acid, lauric acid, and myristic acid.

With respect to the alkali metal salts of linear fatty acids having 16 or more carbon atoms, alkali metal salts of linear fatty acids having 18 or more carbon atoms such as stearic acid and oleic acid are preferable and potassium salts are particularly preferable. With respect to the alkali metal salts of linear fatty acids having 10 to 14 carbon atoms, sodium salt of lauric acid having 12 carbon atoms and potassium salt of myristic acid having 14 carbon atoms are preferable in terms of the dispersibility.

If the content of the linear fatty acids having 16 or more carbon atoms in the composition of the linear fatty acid alkali metal salts is lower than 50% by weight, the dispersibility of the inorganic particles in the resin is slightly worsened as compared with that in the case the content is 50% by weight or higher, although the reason is not so clear, and if the content exceeds 98% by weight, the voids formed between the resin and particles tend to be undesirably small as compared with those in the case the content is 98% by weight or lower. If the voids are too small, in the case the inorganic particles are used for a light reflection film, the resin is undesirably made too thin in the film and easy to deteriorate by a presently available technique, and in the case the inorganic particles are used for a separator film, good ion permeability cannot be guaranteed and therefore, it is not preferable.

If the content of the linear fatty acids having 10 to 14 carbon atoms in the fatty acid composition is lower than 1.5% by weight, the addition effect is insufficient as compared with the effect in the case the content is 1.5% by weight or higher and consequently results in the same consequence as that in the case the content of the linear fatty acids having 18 or more carbon atoms exceeds 98% by weight and the voids undesirably become small. On the contrary, if the content exceeds 50% by weight, the affinity with the resin is lowered as compared with that in the case the content is 50% by weight or lower and problems such as whitening phenomenon and bleeding to the resin surface after molding tend to be caused and therefore it is not preferable.

In the case the above-mentioned alkali metal salts of the linear fatty acids are used as the surfactant (A), it is preferable to select, mix, and adjust the respective fatty acid compositions, but commercialized soap with a similar composition, for example, Nonsoul SK-1 (®, manufactured by Nippon Oil & Fats Co., Ltd.) may be used to an extent that the effect of the present invention is not interfered.

The use amount of the surfactant (A) is changed in accordance with the specific surface area of the inorganic particles and generally, the use amount is increased more as the inorganic particles with higher specific surface area are used.

However, although it is difficult to generalize it clearly since it also changes in accordance with the physical properties such as MI value of the resin to be the substrate of a porous film and the conditions of active agents to be added at the time of compounding, the use amount is generally 0.1% by weight or higher and 20% by weight or lower to inorganic particles. If the use amount is lower than 0.1% by weight, no sufficient dispersion effect is obtained and on the other hand, if it exceeds 20% by weight, the bleeding of the surfactant (A) to the porous film surface and decrease of the strength of the porous film may become problems.

The use amount of the surfactant (A) in the present invention is proportional to the specific surface area Swx of the inorganic particles to be surface-treated, and it is made clear that if the surfactant (A) is used in a range of ±20% from the amount defined by the following equation (1), the effect of the present invention is more preferably provided.

[use amount (%) of surfactant (A) to inorganic particles]=⅓×[BET specific surface area Swx of inorganic particles before surface treatment] (1)

Examples of the compound (B) having chelating function to an alkaline earth metal to be used in the present invention may be aminocarboxylic acid type chelating agents such as ethylenediamine tetraacetic acid, nitrilotriacetic acid, hydroxyethylethylenediamine triacetic acid, diethylenetriamine pentaacetic acid, and triethylenetetramine hexaacetic acid; phosphonic acid type chelating agents such as hydroxyethylidene diphosphorous acid and nitrilotrismethylene phosphonic acid; aluminum compound-based water treatment agents such as polyaluminum chloride; polycarboxylic acids such as polyacrylic acid and citric acid and their salts; salts of copolymers of maleic acid or itaconic acid with polyacrylic acid; phosphoric acids such as polyphosphoric acid and condensed phosphoric acid and their salts.

Examples of the salts of polycarboxylic acids are polysodium acrylate and polyammonium acrylate: examples of the salts of copolymers are ammonium salts of acrylic acid-maleic acid copolymers (polymerization ratio 100:80 or the like) and ammonium salts of acrylic acid-methacrylic acid copolymers (polymerization ratio 100:80 or the like): examples of the salts of phosphoric acids are sodium hexametaphosphate, sodium polyphosphate, and sodium pyrophosphate. These salts may be use alone or if necessary two or more of them may be used in combination.

In the present invention, with respect to these compounds (B) having the chelating function to an alkaline earth metal, in the case highly advanced insulation property is required just like the case of a secondary lithium battery, polyphosphoric acid, condensed phosphoric acid, polycarboxylic acid, and their salts are preferable and a cyclic condensed phosphoric acid as a phosphoric acid or metaphosphoric acid are especially preferable.

As described in the explanation of the surfactant (A), although it is difficult to clearly generalize the use amount of the compound (B) having chelating function to an alkaline earth metal since it also changes in accordance with the specific surface area of inorganic particles, the resin to be used, and the compounding conditions, the use amount is generally preferable to be 0.05% by weight or higher and 7% by weight or lower to inorganic particles. If the use amount is lower than 0.05% by weight, no sufficient dispersion effect is obtained and on the other hand, if it exceeds 7% by weight, no further improvement of the addition effect is expected and therefore it is not preferable.

The use amount of the compound (B) having chelating function to an alkaline earth metal is proportional to the specific surface area Swx of the inorganic particles to be surface-treated, and it is made clear that if the compound (B) is used in a range of ±20% from the amount defined by the following equation (2), the effect of the present invention is more preferably obtained.

[use amount (%) of compound (B) to inorganic particles]= 1/9×[BET specific surface area Swx of inorganic particles before surface treatment] (2).

The inorganic particles to be used in the present invention are not particularly limited if they are generally insoluble in water and preferable examples are those which contain alkaline earth metals as main components, auxiliary components, or impurities such as barium sulfate, calcium carbonate, basic magnesium carbonate, magnesium hydroxide, hydroxytalcite, hydroxy apatite, talc, and clay. Especially, calcium carbonate, basic magnesium carbonate, magnesium hydroxide, hydroxytalcite, and hydroxy apatite are preferable.

Among them are barium sulfate and calcium carbonate preferable since they are safe, economically available, and relatively easy to adjust the particle diameter and scarcely contain impurities which are easy to remove. Particularly, calcium carbonate is more preferable since the entire process in its production is safe and high quality of limestone is produced domestically and its raw materials are thus abundant.

In general, calcium carbonate is broadly classified into two types: one is heavy calcium carbonate obtained by mechanically crushing limestone and classifying and adjusting the crushed fragments in various grades and precipitated calcium carbonate (synthesized calcium carbonate) produced by chemical methods such as a carbon dioxide gas synthesizing method involving causing reaction of quick lime obtained by firing limestone at a high temperature and water for obtaining lime milk, and introducing carbon dioxide gas generated at the time of firing the limestone into the lime milk, a lime-sodium carbonate method involving reaction of sodium carbonate with lime milk, or a calcium chloride-sodium carbonate method involving reaction of calcium chloride and sodium carbonate.

If the surface-treated calcium carbonate satisfies the above-mentioned conditions of the present invention, there is no physical property difference due to the difference of the production methods, however since limestone, which is a raw material of heavy calcium carbonate, contains impurities consisting of various elements other than calcium carbonate and derived from the production process, heavy calcium carbonate is not preferable in use for a separator of batteries which requires calcium carbonate with high purity and undesirable to contain such impurities. Further, heavy calcium carbonate is not preferable also from a viewpoint that calcium carbonate whose particle size distribution is comprehensively broad and that it is impossible to produce calcium carbonate with fine particle size to a prescribed level or higher by presently available crushing and classifying techniques.

With respect to the lime-sodium carbonate method involving reaction of lime milk and sodium carbonate and the sodium method involving reaction of calcium chloride and sodium carbonate, the precipitated calcium carbonate to be obtained by these methods are advantageous for a separator of a battery since the particle size is in a sharp distribution range and easy to be adjusted and the impurities contained in the calcium carbonate are extremely slight.

However, raw materials of the heavy calcium carbonate and precipitated calcium carbonate produced by the carbon dioxide gas synthesis method are only limestone and coke and light oil to be used for firing and on the other hand, in the method of using sodium carbonate, limestone and salts are used as starting raw materials to industrially produce sodium carbonate and calcium chloride, which are raw materials, and it is not preferable to obtain calcium carbonate again using these raw materials in terms of the load on environments, which is a today's hot issue, even in the case the raw materials can be obtained in advantageous conditions in terms of the cost.

Further, in the case good dispersibility is required just like the case of the particles of the present invention, it is needed to remove a counter ion and therefore a large quantity of water is needed to wash the particles after reaction and accordingly, the method is not preferable in terms of the cost and the load on environments.

In the case of the precipitated calcium carbonate obtained by firing limestone for obtaining quick lime and causing reaction of lime milk obtained by dissolving the obtained quick lime and carbon dioxide gas obtained at the time of firing, the method gives fine particles with uniform particle diameter and shape in form of primary particles and scarcely containing impurities, and also the method is suitable for adjusting the particle size and removing coarse particles by adjusting the reaction conditions and post-process after the reaction and excellent in the economical properties in relation to the physical properties of the particles to be obtained and in the load on the environments, and accordingly preferable for using the particles for a film for a battery separator. In the case the particles are used for a battery separator, the limestone, which is a raw material, is preferable to be selected in consideration of impurities and as a fuel for firing the raw material, generally coke and light oil are used, however in terms of the impurities, firing is preferable to be carried out using light oil as long as the cost allows.

Further, the calcium carbonate particles obtained by the reaction are preferable to be subjected to removal of impurities and coarse particles by gravitational separation such as decantation and centrifugation, classification using buoyant (density) separation, and sieves and filters in the state the particles are in a water slurry form.

The calcium carbonate obtained by drying and crushing or surface-treated calcium carbonate powder is also preferable to be subjected to classification treatment such as air blowing classification to remove agglomerates formed by drying.

The surface treatment method for the calcium carbonate particles using the above-mentioned surfactant (A) and compound (B) having chelating function to an alkaline earth metal may be a method, generally so-called dry treatment, involving directly mixing the surface treatment agent with the powder using a mixer such as a Super mixer, a Henshel mixer, or the like and if necessary heating the mixture; a method, generally so-called wet treatment, involving dissolving the surfactant (A) and the compound (B) having a chelating function to an alkaline earth metal in water or hot water, adding the obtained solution to a water slurry containing calcium carbonate under stirring condition, dewatering and drying the obtained mixture; or a combination method of these methods, however, in terms of the extent of the treatment of the calcium carbonate particle surface and economy, mainly the wet treatment is preferable to be employed alone.

The surface-treated inorganic particles in the present invention are preferable to satisfy the following particle size properties (1) to (4):

0.3≦D₅₀≦1.5 [μm]; (1)

0.02≦Dₓ≦0.6 [μm]; (2)

Dₐ≦20 [μm]; and (3)

3≦Sw≦60 [m²/g] (4)

wherein,

D₅₀: average particle diameter [μm] of on-sieve particles in cumulative distribution measured by Microtrac FRA manufactured by Leeds & Northrup;

Dₓ: average particle diameter [μm] of particles left after observation of randomly selected 100 particles using a scanning electron microscope at 20,000 times magnification and elimination of the maximum and minimum 20 particles each among the particles;

Dₐ: maximum particle size [μm] observed in the case of measurement by Microtrac FRA manufactured by Leeds & Northrup; and

Sw: BET specific surface area [m²/g] measured by a nitrogen adsorption method.

The surface-treated inorganic particles of the present invention have an average particle diameter D₅₀ measured by Microtrac FRA manufactured by Leeds & Northrup preferably in a range of 0.3≦D₅₀≦1.5 [μm] and more preferably in a range of 0.3≦D₅₀≦1.0 [μm].

It is technically possible to make the average particle diameter D₅₀ smaller than 0.3 μm, however it is undesirable in terms of the cost and if D₅₀ exceeds 1.5 μm, cohesion of secondary particles composed of agglomerates of primary particles becomes high and a portion of particles may sometimes remain in form of secondary particles in the resin and the particles form voids exceeding the desired size in a porous film for a light reflection layer or a film for a battery separator and therefore, in the case the particles are used, for example, for a light reflection film, the reflected light rays undesirably tend to be uneven, and they are used, for example, for a separator film, the ion permeability undesirably becomes uneven.

The particle diameter Dₓ of the surface-treated inorganic particles of the present invention observed by an electron microscopic observation field is preferable in a range of 0.02≦Dₓ≦0.6 [μm] and more preferable in a range of 0.02≦Dₓ≦0.4 [μm].

If the particle diameter Dₓ exceeds 0.6 μm, in the case the inorganic particles are added to a porous film for a light reflector or a separator film for a battery, voids larger than an aimed size are formed and therefore it is not preferable. On the other hand, if it is smaller than 0.02 μm, the voids formed between the resin and particles tend to become small and therefore it is not preferable and further, cohesion among particles becomes high and a portion of particles may sometimes behave same as coarse particles without being dispersed when being mixed with a resin and in the case the particles are used for a porous film for a light reflector or a separator film for a battery, the particles form voids larger than aimed and therefore, it is not preferable.

The surface-treated inorganic particles of the present invention have the maximum particle diameter Dₐ measured by the above-mentioned Microtrac FRA preferably in a range of Dₐ≦20 [μm] and more preferably in a range of Dₐ≦5 [μm]. Particularly, in the case of using the inorganic particles for a separator film for a battery, Dₐ is further preferably in a range of Dₐ≦3 [μm].

If the maximum particle diameter Dₐ exceeds 20 μm, if being mixed in a porous film for a light reflector or a separator film for a battery, the inorganic particles form voids with a size exceeding the aimed size and therefore, it is not preferable.

A medium to be used for the measurement with Microtrac FRA may be selected properly in accordance with the surface treatment agent used for the surface treatment of the particles, however generally water is preferable to be used for those which are surface-treated with a hydrophilic surface treatment agent, and methanol or ethanol is preferable to be used for those which are surface-treated with a hydrophobic surface treatment agent.

Further, at the time of measurement, the particles are previously dispersed in water or methanol or ethanol in slurry state and subjected to ultrasonic radiation at 300 μA for 60 seconds using a Ultra Sonic Generator US-300T manufactured by Nihonseiki Kaisha Ltd. and then measurement is carried out.

The surface-treated inorganic particles of the present invention have a BET specific surface area Sw measured by a nitrogen adsorption method preferably in a range of 3≦Sw≦60 [m²/g] and more preferably in a range of 5≦Sw≦20 [m²/g].

If the BET specific surface area Sw exceeds 60 m²/g, as described above, the voids tend to become small and particles tend to be agglomerated and therefore, it is undesirable in terms of the dispersibility and if it is lower than 3 m²/g, the primary particles become too large and in the case the inorganic particles are mixed in a porous film for a light reflector or a separator film for a battery, the particles form voids larger than aimed size and therefore the particles are unsuitable for particles to be used for a backlight apparatus or a secondary lithium battery.

The filler for porous films comprising the surface-treated inorganic particles obtained in the above-mentioned manner is mixed with various kinds of resins, particularly olefin type resins and used for producing porous films for various purposes, particularly for light reflectors and battery separators.

A resin to be used in the present invention is not particularly limited, however examples of the resin may include polyester, polycarbonate, polyethylene, polypropylene, ethylene-propylene copolymers, and copolymers of ethylene or propylene with other monomers.

In the case of using the resin as a porous film for light reflection layers, polyolefin type resins such as polyethylene and polypropylene are preferable and particularly polypropylene is more preferable since the brightness decrease as described above is low and stable with the lapse of time, and the resin itself has flexibility and hardly scratches a light guide plate.

Further, in the case of using the resin as a separator film for batteries, polyolefin type resins such as polyethylene and polypropylene are preferable and particularly polyethylene is more preferable since the above-mentioned shut down mechanism is provided and such resins are advantageous in handling property at the time of battery production and cost.

The mixing ratio of the filler for porous films and these resins is not particularly limited and largely differs in accordance with the types and use of the resins, desired physical properties and the cost and may be appropriately selected based on these factors, however the ratio is generally 60 to 150 parts by weight and preferably about 80 to 120 parts by weight to 100 parts by weight of the resin.

To an extent that the effects of the filler for porous films of the present invention are not interfered, in order to improve the film characteristics, a lubricant such as fatty acids, fatty acid amides, ethylene bisstearic acid amide, and sorbitan fatty acid esters, a plasticizer, a stabilizer, and an antioxidant may be added and further, additives used commonly for resin compositions for films such as a lubricant, an antioxidant, a heat stabilizer, a photo-stabilizer, a ultraviolet absorbent, a neutralizing agent, an antifogging agent, an anti-blocking agent, an antistatic agent, a slipping agent, a coloring agent or the like may be added.

In the case the filler for porous films of the present invention and the above-mentioned various kinds of additives are mixed with a resin, generally, the resin mixture is heated and kneaded using a uniaxial or biaxial extruder, a kneader, or a Bumbury's mixer and a sheet is formed using a T die and successively the sheet is stretched uniaxially or biaxially to obtain a porous film product having fine pores.

Further, film formation is carried out using a conventionally known molding apparatus for T-die extrusion or inflation formation after kneading and the obtained films may be treated with an acid for dissolving the filler for porous films of the present invention to obtain a porous film product having fine pores.

As the shape of the resin, there are pellet type and powder (granular) type with adjusted particle diameter and it is preferable to use a powder type resin for particle dispersion and mix them by a conventionally known mixing apparatus, so-called mixer, such as a Henshel mixer, a tumbler type mixer, and a ribbon blender.

The filler for porous films of the present invention shows good physical properties such as dispersibility in the resin as compared with particles other than the present invention, even when the filler is used with a pellet type resin, however the filler is particularly preferable to be used while being mixed with a powder resin and in the case of mixing the filler and the resin by a Henshel mixer, the mixing can be carried out quickly and additionally, the mixing has the following advantageous characteristics: that is, adhesion of the mixture to the inner wall face and the mixing and stirring blades of the mixer is little: deformation of the resin due to the adhesion to the inside of the mixer and formation of agglomerates scarcely occur: the workability of the mixing becomes excellent: and occurrence of clogging of the strainer in a kneading extruder in the post-treatment is suppressed.

There are various types of the above-mentioned heating and kneading apparatus and setting conditions and a raw material loading method may be properly determined in consideration of the dispersion of the particles in the resin as well as the effect of the resin itself on the MI value and the cost. In the case the filler for porous films of the present invention is blended with the resin, the above-mentioned matter is also taken into consideration to select the types and conditions, however it is preferable to quantitatively loading a mixture of the filler mixed with the resin powder having a particle size within a proper range by a Henshel mixer or the like to a hopper of a kneader such as a biaxial kneader.

Pellets containing various kinds of additives represented by the filler for porous films of the present invention, so-called as a master batch, may be produced between a mixing apparatus and film formation and thereafter melting and film formation may be carried out after mixing with of a resin containing no additive. If necessary, a plurality of T-die extruders are layered during the above-mentioned process or a lamination process may be introduced during the stretching to produce a multilayer film. Further, in order to provide printability to the above-mentioned film, an ink receiving layer may be formed after surface treatment of the film surface by plasma discharge or the like is carried out.

### EXAMPLES

Hereinafter, the present invention will be described with reference to Examples, however it is not intended that the present invention be limited to the illustrated Examples.

In the following descriptions, % means % by weight unless otherwise specified.

### Example 1

After foreign matter removal by a sieve, quick lime obtained by firing gray and dense limestone by a fluidized bed type kiln using kerosene as a heat source was dissolved in water to obtain a slaked lime slurry and after further removal of foreign matter and coarse particles by a cyclone, the slurry was reacted with carbon dioxide gas and thereafter, elution of particles of calcium carbonate in water and adsorption of the particles are repeated, that is, so-called Ostwald aging was carried out, to grow the particles and obtain a water slurry containing 10% of precipitated calcium carbonate with a BET specific surface area of 10 m²/g.

Next, using a separately produced mixed treatment agent A1 having the following composition as a surfactant (A), an aqueous solution of the surfactant (A) was produced by dissolving the agent A1 at a ratio of 3.3% to the solid matter of the calcium carbonate in hot water at 80° C. and further hexametaphosphoric acid sodium salt (first grade reagent) as the compound (B) having chelating function to an alkaline earth metal (hereinafter, referred to as the chelating compound) was dissolved at a ratio of 0.9% to solid matter of the calcium carbonate in water at 40° C. to produce an aqueous solution of the chelating compound (B).

While the previously obtained precipitated calcium carbonate slurry was stirred and adjusted to 60° C., the above-mentioned chelating compound (B) and surfactant (A) were successively added and the mixture was stirred for 4 hours to obtain a surface treatment calcium carbonate slurry.

The obtained surface treatment calcium carbonate slurry was subjected to foreign matter and coarse particle removal by a high speed decanter manufactured by TANABEWILLTEC Co., Ltd. and a sieve with a 350 mesh and further dewatered, dried, and pulverized. The obtained dry powder was further classified by an air classifying apparatus to obtain a surface-treated calcium carbonate powder.

The obtained surface-treated calcium carbonate powder had D₅₀ of 0.476 μm, Dₓ of 0.15 μm, Dₐ of 1.635 μm, and Sw of 9.3 m²/g.

Mixed treatment agent A1:
potassium stearate 65%,
sodium palmitate 20%, and
sodium laurate 15%

### Example 2

The same process was carried out as that of Example 1 to obtain a surface-treated calcium carbonate powder, except that the surfactant (A) was changed to potassium stearate. The physical properties of the obtained surface-treated calcium carbonate powder are shown in Table 1.

### Example 3

The same process was carried out as that of Example 1 to obtain a surface-treated calcium carbonate powder, except that the surfactant (A) was changed to sodium laurate. The physical properties of the obtained surface-treated calcium carbonate powder are shown in Table 1.

### Example 4

The same process was carried out as that of Example 1 to obtain a surface-treated calcium carbonate powder, except that the surfactant (A) was changed to sodium oleate. The physical properties of the obtained surface-treated calcium carbonate powder are shown in Table 1.

### Example 5

The same process was carried out as that of Example 1 to obtain a surface-treated calcium carbonate powder, except that the surfactant (A) was changed to sodium abietate. The physical properties of the obtained surface-treated calcium carbonate powder are shown in Table 1.

### Example 6

The same process was carried out as that of Example 1 to obtain a surface-treated calcium carbonate powder, except that the surfactant (A) was changed to lauric acid. The physical properties of the obtained surface-treated calcium carbonate powder are shown in Table 1.

### Example 7

The same process was carried out as that of Example 1 to obtain a surface-treated calcium carbonate powder, except that the chelating compound (B) was changed to polyaluminum chloride. The physical properties of the obtained surface-treated calcium carbonate powder are shown in Table 1.

### Example 8

The same process was carried out as that of Example 1 to obtain a surface-treated calcium carbonate powder, except that the chelating compound (B) was changed to polysodium acrylate. The physical properties of the obtained surface-treated calcium carbonate powder are shown in Table 1.

### Example 9

The same process was carried out as that of Example 1 to obtain a surface-treated calcium carbonate powder, except that the chelating compound (B) was changed to nitrilotriacetic acid. The physical properties of the obtained surface-treated calcium carbonate powder are shown in Table 1.

### Example 10

The same process was carried out as that of Example 1 to obtain a surface-treated calcium carbonate powder, except that the chelating compound (B) was changed to hydroxyethylidene diphosphorous acid. The physical properties of the obtained surface-treated calcium carbonate powder are shown in Table 1.

### Example 11

The same process was carried out as that of Example 1 to obtain a surface-treated calcium carbonate powder, except that the chelating compound (B) was changed to a polyacrylic acid-maleic acid copolymer (weight ratio 100:80). The physical properties of the obtained surface-treated calcium carbonate powder are shown in Table 1.

### Example 12

The same process was carried out as that of Example 1 to obtain a surface-treated calcium carbonate powder, except that the addition amount of the surfactant (A) was changed to 5%. The physical properties of the obtained surface-treated calcium carbonate powder are shown in Table 1.

### Example 13

The same process was carried out as that of Example 1 to obtain a surface-treated calcium carbonate powder, except that the addition amount of the chelating compound (B) was changed to 2%. The physical properties of the obtained surface-treated calcium carbonate powder are shown in Table 1.

### Examples 14 to 18

Water slurries containing 10% of precipitated calcium carbonate were obtained in the same manner as Example 1, except that the particle growth by aging was stopped to adjust BET specific surface area Swx cm²/g and that addition amounts of the surfactants (A) and chelating compounds (B) were changed as shown in Table 2 and successively the same process was carried out as that of Example 1 to obtain surface-treated calcium carbonate powders. The respective physical properties of the obtained surface-treated calcium carbonate powders are shown in Table 1.

### Example 19

The same process was carried out as that of Example 1 to obtain a surface-treated calcium carbonate powder, except that no aging was carried out after the reaction and that the addition amounts of the surfactant (A) and the chelating compound (B) were changed to 20% and 7%, respectively. The physical properties of the obtained surface-treated calcium carbonate powder are shown in Table 1.

### Example 20

The same process was carried out as that of Example 1 to obtain a surface-treated calcium carbonate powder, except that the surfactant (A) was changed to a commercialized soap (Nonsoul SK-1®, manufactured by Nippon Oil & Fats Co., Ltd.) and that the chelating compound (B) was changed to sodium hexametaphosphate for industrial use. The physical properties of the obtained surface-treated calcium carbonate powder are shown in Table 1.

The typical composition of the used soap is as shown below.

Nonsoul SK-1:

| | |
|---|---|
| potassium palmitate | 27.4% |
| potassium stearate | 65.6% |
| potassium arachidate | 1.4% |
| potassium behenate | 1.0% |
| potassium myristate | 2.0% |
| others | 2.6% |

### Example 21

According to a method described in JP-A No. 7-196316, 100 L of a 1.5 mol/L sodium carbonate solution, 100 L of a 1.35 mol/L calcium chloride solution, and a 0.04 mol/L sodium hydroxide solution were prepared and the sodium carbonate solution and the sodium hydroxide solution were mixed and the mixed solutions and the calcium chloride solution were respectively adjusted to 16.0° C.

Under stirring condition, 100 L of the calcium chloride solution was added dropwise for 200 seconds to 200 L of the mixed solution of the sodium carbonate solution and sodium hydroxide solution and after 180 seconds from completion of the dropwise addition, sodium hexametaphosphate (first grade reagent) in an amount equivalent to 0.8 wt % of calcium carbonate to be theoretically produced by the reaction was added and the obtained reaction slurry was stirred further for 5 minutes.

The slurry containing the calcium carbonate surface treated with sodium hexametaphosphate was subjected to dewatering and dilution treatment by a high speed decanter to remove a counter ion and foreign matter and after that, the slurry was adjusted to 60° C. and successively, after the mixed treatment agent A1 in an amount of 2.9% to the calcium carbonate solid matter was dissolved in hot water at 80° C., the obtained solution was added to the calcium carbonate slurry and stirred for 4 hours to obtain a surface-treated calcium carbonate slurry.

The obtained surface-treated calcium carbonate slurry was dried and crushed and further the obtained dry powder was classified by an air classifying apparatus to obtain a surface-treated calcium carbonate powder. The physical properties of the obtained surface-treated calcium carbonate powder are shown in Table 1.

### Example 22

The same process was carried out as that of Example 1 to obtain a surface-treated calcium carbonate powder, except that coke was used as the heat source, that the gray and dense limestone was fired by a shaft type kiln and that no foreign substance removal was carried out. The physical properties of the obtained surface-treated calcium carbonate powder are shown in Table 1.

### Example 23

After a 10% slurry was produced by mixing a white marble-like limestone, the slurry was wet crushed by a wet crusher DINO-MILL KB-20B to obtain a water slurry containing calcium carbonate with a BET specific surface area of 0.9 m²/g.

Next, the mixed treatment agent A1 in an amount of 0.3% to the calcium carbonate solid matter was dissolved in hot water at 80° C. to produce an aqueous solution of the surfactant (A). Further, sodium hexametaphosphate (first grade reagent) in an amount of 0.1% to the calcium carbonate solid matter was dissolved in water at 40° C. to produce an aqueous solution of the chelating compound (B).

While the previously produced calcium carbonate slurry was stirred and adjusted to 60° C. and the above-mentioned chelating compound (B) and the surfactant (A) were successively added to the slurry and stirred for 4 hours to obtain a surface-treated calcium carbonate slurry.

The obtained surface treatment calcium carbonate slurry was subjected to foreign matter and coarse particle removal by a high speed decanter manufactured by TANABEWILLTEC Co., Ltd. and a sieve with a 350 mesh and further dewatered, dried, and pulverized. The obtained dry powder was further classified by an air classifying apparatus to obtain a surface-treated calcium carbonate powder.

The physical properties of the obtained surface-treated calcium carbonate powder are shown in Table 1.

### Example 24

While 100 L of 0.8 mol/L barium sulfide solution adjusted to 15° C. was stirred, 100 L of 0.8 mol/L sodium sulfate solution adjusted to 14.4° C. was added dropwise for 400 seconds and mixed to produce barium sulfate.

Next, the mixed treatment agent A1 in an amount of 1.2% to the barium sulfate solid matter was dissolved in hot water at 80° C. to produce an aqueous solution of the surfactant (A). Further, sodium hexametaphosphate (first grade reagent) in an amount of 0.38% to the barium sulfate solid matter was dissolved in water at 40° C. to produced an aqueous solution of the chelating compound (B).

While the previously produced barium sulfate slurry was stirred and adjusted to 60° C. and the above-mentioned chelating compound (B) and the surfactant (A) were successively added to the slurry and stirred for 4 hours to obtain a surface-treated barium sulfate slurry.

The obtained surface-treated barium sulfate slurry was subjected to foreign matter and coarse particle removal by a sieve with a 350 mesh and further dewatered, dried, and pulverized. The obtained dry powder was further classified by an air classifying apparatus to obtain a surface-treated barium sulfate powder.

The physical properties of the obtained surface-treated barium sulfate powder are shown in Table 1.

### Comparative Example 1

The same process was carried out as that of Example 1 to obtain a surface-treated calcium carbonate powder, except that no chelating compound (B) was used as the treatment agent. The physical properties of the obtained surface-treated calcium carbonate powder are shown in Table 1.

### Comparative Example 2

The same process was carried out as that of Example 1 to obtain a surface-treated calcium carbonate powder, except that no surfactant (A) was used as the treatment agent. The physical properties of the obtained surface-treated calcium carbonate powder are shown in Table 1.

TABLE 1

| | Example 1 | Example 2 | Example 3 | Example 4 | Example 5 | Example 6 | Example 7 | Example 8 |
|---|---|---|---|---|---|---|---|---|
| Surfactant (A) | Surfactant A1 | potassium | sodium laurate | sodium oleate | sodium abietate | lauric acid | Surfactant A1 | Surfactant A1 |
| | | stearate | | | | | | |
| Addition amount (%) | 3.3 | 3.3 | 3.3 | 3.3 | 3.3 | 3.3 | 3.3 | 3.3 |
| Chelating compound (B) | sodium | sodium | sodium | sodium | sodium | sodium | polyaluminium | polysodium |
| | hexameta- | hexameta- | hexameta- | hexameta- | hexameta- | hexameta- | chloride | acrylate |
| | phosphate | phosphate | phosphate | phosphate | phosphate | phosphate | | |
| Addition amount (%) | 0.9 | 0.9 | 0.9 | 0.9 | 0.9 | 0.9 | 0.9 | 0.9 |
| BET of inorganic | 10 | 10 | 10 | 10 | 10 | 10 | 10 | 10 |
| particles Swx | | | | | | | | |
| Average particle | 0.476 | 0.544 | 0.638 | 0.495 | 0.868 | 0.714 | 0.621 | 0.481 |
| diameter D₅₀ | | | | | | | | |
| Average particle | 0.15 | 0.15 | 0.15 | 0.15 | 0.15 | 0.15 | 0.15 | 0.15 |
| diameter by electron | | | | | | | | |
| microscope | | | | | | | | |
| observation Dx | | | | | | | | |
| Maximum particle size | 1.635 | 1.945 | 1.635 | 1.945 | 2.75 | 2.75 | 2.313 | 1.635 |
| Da | | | | | | | | |
| BET specific surface | 9.3 | 9.2 | 9.5 | 9.2 | 9.2 | 9.4 | 9.1 | 9.4 |
| area Sw | | | | | | | | |

| | | | | | | Example | Example | Example |
|---|---|---|---|---|---|---|---|---|
| | Example 9 | Example 10 | Example 11 | Example 12 | Example 13 | 14 | 15 | 16 |
| Surfactant (A) | Surfactant A1 | Surfactant A1 | Surfactant A1 | Surfactant A1 | Surfactant A1 | Surfactant | Surfactant | Surfactant |
| | | | | | | A1 | A1 | A1 |
| Addition amount (%) | 3.3 | 3.3 | 3.3 | 5 | 3.3 | 5 | 5 | 1.9 |
| Chelating compound (B) | nitrilotriacetic | hydroxyethylidene | acrylic acid- | sodium | sodium | sodium | sodium | sodium |
| | acid | diphosphorous | maleic acid | hexameta- | hexameta- | hexameta- | hexameta- | hexameta- |
| | | acid | copolymer | phosphate | phosphate | phosphate | phosphate | phosphate |
| Addition amount (%) | 0.9 | 0.9 | 0.9 | 0.9 | 2 | 1.7 | 1.2 | 0.67 |
| BET of inorganic | 10 | 10 | 10 | 10 | 10 | 15 | 15 | 5.6 |
| particles Swx | | | | | | | | |
| Average particle | 0.775 | 0.544 | 0.847 | 0.687 | 0.467 | 1.385 | 1.84 | 0.97 |
| diameter D₅₀ | | | | | | | | |
| Average particle | 0.15 | 0.15 | 0.15 | 0.15 | 0.15 | 0.07 | 0.07 | 0.52 |
| diameter by electron | | | | | | | | |
| microscope | | | | | | | | |
| observation Dx | | | | | | | | |
| Maximum particle size | 2.313 | 1.945 | 2.313 | 2.313 | 1.635 | 2.75 | 2.75 | 2.313 |
| Da | | | | | | | | |
| BET specific surface | 9.2 | 9.4 | 9.2 | 9.3 | 9.1 | 14 | 14 | 5.1 |
| area Sw | | | | | | | | |

| | Example 17 | Example 18 | Example 19 | Example 20 | Example 21 | Example 22 | Example 23 | Example 24 |
|---|---|---|---|---|---|---|---|---|
| Surfactant (A) | Surfactant | Surfactant | Surfactant | SK-1 | Surfactant | Surfactant A1 | Surfactant A1 | Surfactant A1 |
| | A1 | A1 | A1 | | A1 | | | |
| Addition amount (%) | 7 | 5 | 20 | 3.3 | 2.9 | 3.3 | 0.3 | 1.2 |
| Chelating compound (B) | sodium | sodium | sodium | sodium | sodium | sodium | sodium | sodium |
| | hexameta- | hexameta- | hexameta- | hexameta- | hexameta- | hexameta- | hexameta- | hexameta- |
| | phosphate | phosphate | phosphate | phosphate | phosphate | phosphate | phosphate | phosphate |
| | | | | (for industry) | | | | |
| Addition amount (%) | 1.9 | 1.5 | 7 | 0.9 | 0.8 | 0.9 | 0.1 | 0.38 |
| BET of inorganic | 21 | 21 | 58 | 10 | 3.2 | 10 | 0.9 | 3.4 |
| particles Swx | | | | | | | | |
| Average particle | 0.74 | 0.94 | 0.98 | 0.482 | 0.61 | 0.524 | 0.98 | 0.783 |
| diameter D₅₀ | | | | | | | | |
| Average particle | 0.03 | 0.03 | 0.02 | 0.15 | 0.62 | 0.15 | 0.94 | 0.54 |
| diameter by electron | | | | | | | | |
| microscope Dx | | | | | | | | |
| Maximum particle size Da | 4.625 | 18.5 | 2.313 | 1.945 | 2.313 | 22 | 9.25 | 5.5 |
| BET specific surface | 19.5 | 19.5 | 56 | 9.1 | 3 | 9.3 | 0.9 | 3.2 |
| area Sw | | | | | | | | |

| | Comparative Example 1 | Comparative Example 2 |
|---|---|---|
| Surfactant (A) | Surfactant A1 | absence |
| Addition amount (%) | 5 | — |
| Chelating compound (B) | absence | sodium hexametaphosphate |
| Addition amount (%) | — | 1.67 |
| BET of inorganic particles Swx | 10 | 10 |
| Average particle diameter D₅₀ | 1.382 | 0.422 |
| Average particle diameter | 0.15 | 0.15 |
| by electron microscope | | |
| observation Dx | | |
| Maximum particle size Da | 15.56 | 1.635 |
| BET specific surface area Sw | 8.9 | 9.3 |

### Examples 25 to 48 and Comparative Examples 3 and 4

Each filler-resin mixture was obtained by mixing 100 parts of polypropylene resin (FS2011DG2, manufactured by Sumitomo Chemical Industry Co., Ltd., MI=2.0 g/10 min), 110 parts of a filler for a light reflection porous film containing each surface treated calcium powder obtained in Examples 1 to 24 and Comparative Examples 1 and 2, and 1 part of calcium stearate for 5 minutes by a Henshel mixer.

The obtained mixture was pelletized by a bent type biaxial extruder. An un-stretched sheet was obtained from the obtained pellets using an extruder equipped with a T die. The obtained un-stretched sheet was stretched about 7 times as large at 140° C. in a tenter oven to obtain each 180 μm-thick stretched porous film.

The obtained stretched porous film was coated with a polyester type hot melt adhesive in 7 μm thickness by a gravure coater. The stretched porous film coated with the adhesive was laminated on a 200 μm-thick aluminum film, which is a sheet-like support, at a temperature of 75° C. to obtain a light reflection plate. The adhesion strength was 100 g/cm².

Each light reflection plate obtained in such a manner was subjected to measurements and evaluations of total light reflectance, brightness unevenness, color tone alteration (yellowing) in the case of continuous lighting. The results are shown in Table 2.

The total light reflectance was measured according to JIS-Z-8701 by calculating the average values of the reflectance measured in a wavelength range from 40 nm to 700 nm.

Further, each light reflection plate was subjected to the following high temperature environmental test (durability test) to measure the alteration ratio of the total light reflectance (%) {[(total light reflectance before durability test−total light reflectance after durability test)/total light reflectance before durability test]×100}.

Each light reflection plate was left at 83° C. and 50% relative humidity for 24 hours.

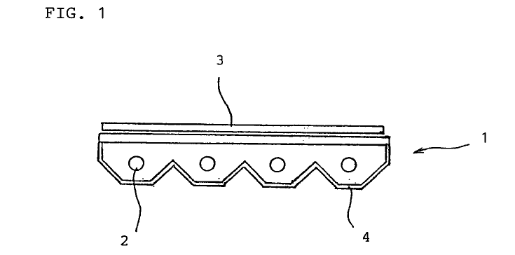

The brightness unevenness was evaluated using an immediate under type plane light source display apparatus with 24 inch size as shown in FIG. 1. Each light reflection plate obtained in Examples 25 to 48 and Comparative Examples 3 and 4 and formed to be a light reflector 4 for a plane light source display apparatus was employed for the apparatus and a cold cathode lamp 2 and LCD cell 3 were installed in the inside and the front of a housing 1, respectively.

Whether brightness unevenness was caused or not was observed with eyes when the apparatus was turned on and light was radiated and the evaluation was carried out based on the following standard.

∘: even brightness and no unevenness

×: uneven brightness

The color tone alteration (yellowing) at the time of continuous lighting was evaluated using Eye Super UV Tester SUV-W 13 (manufactured by IWASAKI ELECTRIC Co., Ltd.). The color tone alteration evaluation was carried out by radiating light at radiation intensity of 90 mW/cm² for 24 hours from a metal halide lamp set at 10 cm distance from the film surface of the light reflector, measuring the color tone alteration of the film by a colorimeter (S & M Color Computer, manufactured by Suga Test Instruments Co., Ltd.) before and after the light radiation test, reading color difference and EH values (JIS-Z-8730) from the respective index values, and evaluating the values based on the following standard.

⊙: no color tone alteration was observed and excellent (EH<0.3)

∘: color tone alteration was scarcely observed and good (0.3≦EH<1)

×: color tone was changed and defective (EH≧1)

### Comprehensive Evaluation

The above-mentioned evaluations were comprehensively evaluated. That is, those which were found most excellent were graded to be 5 and the evaluation was carried out according to the following 5-point grades.

5: extremely excellent

4: excellent

3: good,

2: slightly inferior

1: inferior

TABLE 2

| | Example 25 | Example 26 | Example 27 | Example 28 | Example 29 | Example 30 | Example 31 | Example 32 |
|---|---|---|---|---|---|---|---|---|
| Filler used | Example 1 | Example 2 | Example 3 | Example 4 | Example 5 | Example 6 | Example 7 | Example 8 |
| Total light reflectance | | | | | | | | |
| Before durability test [%] | 95.5 | 94.8 | 93.4 | 92.4 | 90.5 | 93.4 | 92 | 94.6 |
| After durability test [%] | 95.3 | 92.6 | 91.1 | 90.5 | 89.9 | 89.8 | 91.3 | 94 |
| Alteration ratio [%] | 0.21 | 2.32 | 2.46 | 2.06 | 0.66 | 3.85 | 0.76 | 0.63 |
| Brightness uneveness [—] | ◯ | ◯ | ◯ | ◯ | ◯ | ◯ | ◯ | ◯ |
| Color tone alteration [symbol] EH [—] | 0.24 | 0.47 | 0.44 | 0.96 | 0.45 | 0.57 | 0.53 | 0.54 |
| Evaluation | ⊚ | ◯ | ◯ | ◯ | ◯ | ◯ | ◯ | ◯ |
| Comprehensive evaluation | 5 | 3 | 3 | 2 | 2 | 3 | 3 | 4 |
| | Example 33 | Example 34 | Example 35 | Example 36 | Example 37 | Example 38 | Example 39 | Example 40 |
| Filler used | Example 9 | Example 10 | Example 11 | Example 12 | Example 13 | Example 14 | Example 15 | Example 16 |
| Total light reflectance | | | | | | | | |
| Before durability test [%] | 91.6 | 92.2 | 92.6 | 95.2 | 94.8 | 94.4 | 92.4 | 93.2 |
| After durability test [%] | 89.8 | 91.2 | 91.4 | 93.9 | 94 | 93.4 | 91.9 | 92.8 |
| Alteration ratio [%] | 1.97 | 1.08 | 1.30 | 1.37 | 0.84 | 1.06 | 0.54 | 0.43 |
| Brightness uneveness [—] | ◯ | ◯ | ◯ | ◯ | ◯ | ◯ | ◯ | ◯ |
| Color tone alteration [symbol] EH [—] | 0.88 | 0.56 | 0.58 | 0.43 | 0.47 | 0.43 | 0.51 | 0.42 |
| Evaluation | ◯ | ◯ | ◯ | ◯ | ◯ | ◯ | ◯ | ◯ |
| Comprehensive evaluation | 2 | 3 | 3 | 4 | 4 | 4 | 3 | 4 |
| | Example 41 | Example 42 | Example 43 | Example 44 | Example 45 | Example 46 | Example 47 | Example 48 |
| Filler used | Example 17 | Example 18 | Example 19 | Example 20 | Example 21 | Example 22 | Example 23 | Example 24 |
| Total light reflectance | | | | | | | | |
| Before durability test [%] | 95.2 | 93.1 | 92.9 | 95.3 | 90.4 | 92.9 | 89.6 | 89.4 |
| After durability test [%] | 94.8 | 92.5 | 92.3 | 95.1 | 90 | 91.6 | 89.4 | 89 |
| Alteration ratio [%] | 0.42 | 0.64 | 0.65 | 0.21 | 0.44 | 1.40 | 0.22 | 0.45 |
| Brightness uneveness [—] | ◯ | ◯ | ◯ | ◯ | ◯ | ◯ | ◯ | ◯ |
| Color tone alteration [symbol] EH [—] | 0.42 | 0.49 | 0.47 | 0.26 | 0.51 | 0.87 | 0.43 | 0.47 |
| Evaluation | ◯ | ◯ | ◯ | ⊚ | ◯ | ◯ | ◯ | ◯ |
| Comprehensive evaluation | 4 | 3 | 3 | 5 | 2 | 2 | 2 | 2 |

| | Comparative Example 3 | Comparative Example 4 |
|---|---|---|
| Filler used | Comparative Example 1 | Comparative Example 2 |
| Total light reflectance | | |
| Before durability test [%] | 88.5 | 83 |
| After durability test [%] | 83.4 | 78.7 |
| Alteration ratio [%] | 5.76 | 5.18 |
| Brightness uneveness [—] | X | X |
| Color tone alteration [symbol] EH [—] | 0.94 | 1.21 |
| Evaluation | ◯ | X |
| Comprehensive evaluation | 1 | 1 |

### Examples 49 to 72 and Comparative Examples 5 and 6

Each filler-resin mixture for porous films was obtained by producing a mixed polyethylene resin by mixing a polyethylene resin (Hi-zex Million 340 M, manufactured by Mitsui Chemicals Inc.) and a polyethylene wax (Hi-wax 110P, manufactured by Mitsui Chemicals Inc.) at 7:3; loading a filler for a light reflection porous film comprising each surface treated calcium powder obtained in Examples 1 to 24 and Comparative Examples 1 and 2, and putting the mixed resin at 3:7 by volume to a Henshel mixer and mixing the mixture for 5 minutes.

The obtained mixture was melted and kneaded by a biaxial kneader 2D25W manufactured by Toyoseiki Co., Ltd. and equipped with a T die and formed into a film to obtain a 80 μm thick film. The obtained film was stretched about 5 times as large at 110° C. in a tenter oven to obtain each porous film.

Each of the obtained porous film was subjected to evaluations of the following various physical properties. The results are shown in Table 3.

### (Evaluation Methods)

### 1) Ion Permeability

The ion permeability was evaluated by measuring the electric conductivity of Li ion moving in a solution. The measurement was carried out by fixing each porous film (previously cut into 47 mm diameter) obtained in the present invention, in place of filtration paper or a filter, between a filter holder to be used in a filtration test or the like and a 250 ml funnel by a clamp and inserting the funnel in a 1 L-capacity suction bottle filled with a mixed solution containing ethylene carbonate, ethyl methyl carbonate, and dimethyl carbonate in a ratio of 30:35:35 by volume, further pouring 200 mL of an electrolytic solution obtained by dissolving 1 mol/L of LiPF₆ as an electrolyte to the mixed solution in the funnel, and measuring the electric conductivity of the electrolytic solution in the suction bottle after 30 minutes. As the value of the electric conductivity was higher, the ion permeability was higher and better.

### 2) Gurley Ventilation

The Gurley value of each porous film was measured using a B-model densometer manufactured by Toyoseiki Co., Ltd. according to JIS-P8117. As the Gurley ventilation value is smaller, the permeability of the gas and ion is higher and preferable.

### 3) Average Fine Pore Diameter

According to ASTM F316-86, the average fine pore diameter was measured by a bubble point method using a Perm-Porometer (manufactured by PMI Co., Ltd.).

### 4) Film Thickness

The film thickness was measured by a film thickness measurement meter. If the thickness is smaller, it is advantageous for the ion permeability, however insulation and the penetration strength between both electrodes become weak. Therefore, those which maintain a good ion permeability and a large thickness are preferable.

### 5) Comprehensive Evaluation

The above-mentioned evaluations were comprehensively evaluated. That is, those which were found most excellent were graded to be 5 and the evaluation was carried out according to the following 5-point grades.

5: extremely excellent

4: excellent

3: good

2: slightly inferior

1: inferior

TABLE 3

| | Example 49 | Example 50 | Example 51 | Example 52 | Example 53 | Example 54 | Example 55 | Example 56 |
|---|---|---|---|---|---|---|---|---|
| Filler used | Example 1 | Example 2 | Example 3 | Example 4 | Example 5 | Example 6 | Example 7 | Example 8 |
| Ion Permeability [μS/cm] | 820 | 670 | 660 | 480 | 510 | 450 | 640 | 720 |
| Gurley ventilation [sec./100 cc] | 70 | 90 | 110 | 150 | 180 | 160 | 90 | 90 |
| Average fine | 0.087 | 0.094 | 0.092 | 0.104 | 0.11 | 0.14 | 0.098 | 0.096 |
| pore diameter [μm] | | | | | | | | |
| Film thickness [μm] | 45 | 44 | 45 | 44 | 46 | 45 | 45 | 45 |
| Comprehensive evaluation | 5 | 3 | 3 | 2 | 2 | 3 | 3 | 4 |
| | Example 57 | Example 58 | Example 59 | Example 60 | Example 61 | Example 62 | Example 63 | Example 64 |
| Filler used | Example 9 | Example 10 | Example 11 | Example 12 | Example 13 | Example 14 | Example 15 | Example 16 |
| Ion Permeability [μS/cm] | 520 | 630 | 590 | 700 | 740 | 720 | 620 | 710 |
| Gurley ventilation [sec./100 cc] | 160 | 140 | 150 | 90 | 100 | 80 | 120 | 90 |
| Average fine | 0.121 | 0.114 | 0.102 | 0.095 | 0.094 | 0.095 | 0.111 | 0.099 |
| pore diameter [μm] | | | | | | | | |
| Film thickness [μm] | 44 | 46 | 47 | 46 | 43 | 44 | 45 | 46 |
| Comprehensive evaluation | 2 | 3 | 3 | 4 | 4 | 4 | 3 | 4 |
| | Example 65 | Example 66 | Example 67 | Example 68 | Example 69 | Example 70 | Example 71 | Example 72 |
| Filler used | Example 17 | Example 18 | Example 19 | Example 20 | Example 21 | Example 22 | Example 23 | Example 24 |
| Ion Permeability [μS/cm] | 710 | 630 | 560 | 810 | 540 | 570 | 520 | 430 |
| Gurley ventilation [sec./100 cc] | 90 | 140 | 130 | 70 | 180 | 180 | 190 | 180 |
| Average fine | 0.094 | 0.098 | 0.095 | 0.082 | 0.121 | 0.141 | 0.145 | 0.19 |
| pore diameter [μm] | | | | | | | | |
| Film thickness [μm] | 45 | 46 | 45 | 43 | 44 | 46 | 45 | 44 |
| Comprehensive evaluation | 4 | 3 | 3 | 5 | 2 | 2 | 2 | 2 |

| | Comparative Example 5 | Comparative Example 6 |
|---|---|---|
| Filler used | Comparative Example 1 | Comparative Example 2 |
| Ion Permeability [μS/cm] | 400 | 360 |
| Gurley ventilation [sec./100 cc] | 220 | 400 |
| Average fine pore diameter [μm] | 0.123 | 0.146 |
| Film thickness [μm] | 44 | 46 |
| Comprehensive evaluation | 1 | 1 |

### INDUSTRIAL APPLICABILITY

As described above, a filler for porous films of the present invention is easy to be mixed with a resin and has good dispersibility in a resin, and accordingly suitable for providing a porous film useful for a light reflector of, for example, a liquid crystal display and a lighting apparatus and a porous film useful for a separator between electrodes of a battery.

1. A filler for porous films comprising inorganic particles surface treated with a surfactant (A) and a compound (B) having a chelating function to an alkaline earth metal.

2. The filler for porous films according to claim 1, wherein the inorganic particles are calcium carbonate or barium sulfate.

3. The filler for porous films according to claim 1, wherein the surfactant (A) is a fatty acid salt.

4. The filler for porous films according to any one of claims 1 to 3, wherein the surfactant (A) has a composition containing 50 to 98% by weight of a linear fatty acid salt having 16 or higher carbon atoms and 1.5 to 50% by weight of a linear fatty acid salt having 10 to 14 carbon atoms.

5. The filler for porous films according to any one of claims 1 to 3, wherein condensed phosphoric acid of the compound (B) having the chelating function to an alkaline earth metal is a cyclic condensed phosphoric acid or metaphosphoric acid.

6. The filler for porous films according to any one of claims 1 to 3, wherein the ratios of the surfactant (A) and the compound (B) having a chelating function to an alkaline earth metal to the inorganic particles are 0.1 to 20% by weight and 0.05 to 7% by weight, respectively.

7. The filler for porous films according to any one of claims 1 to 3 which satisfies the following particle size properties (1) to (4):
0.3≦D₅₀≦1.5 [μm]; (1)
0.02≦Dₓ≦0.6 [μm]; (2)
Dₐ≦20 [μm]; and (3)
3≦Sw≦60 [m²/g] (4)
wherein
D₅₀: average particle diameter [μm] of on-sieve particles in cumulative distribution measured by Microtrac FRA manufactured by Leeds & Northrup;
D_{x:} average particle diameter [μm] of particles left after observation of randomly selected 100 particles using a scanning electron microscope at 20,000 times magnification and elimination of the maximum and minimum 20 particles each among the particles;
Dₐ: maximum particle size [μm] observed in the case of measurement by Microtrac FRA manufactured by Leeds & Northrup; and
Sw: BET specific surface area [m²/g] measured by a nitrogen adsorption method.

8. A porous film containing the filler according to claim 1.

9. The porous film according to claim 8, wherein the resin of the porous film is a polyolefin type resin.

10. The porous film according to claim 8 or 9 to be used for light reflection.

11. The porous film according to claim 8 or 9 to be used for a light reflector for a liquid crystal display apparatus or a lighting apparatus.

12. The porous film according to claim 8 to be used for a separator between electrodes of a battery.

13. The porous film according to claim 9 to be used for a separator between electrodes of a battery.

14. The porous film according to claim 12 or 13, wherein the battery is a secondary lithium battery.
